(19) United States
(12) Patent Application Publication
(10) Pub. No.: US 2025/0273313 A1
(43) Pub. Date: Aug. 28, 2025

(54) HYDRATION FLUID PRESCRIBING SYSTEM

(71) Applicant: RENAL CARE & RESEARCH, Walhain (BE)

(72) Inventor: Agnieszka POZDZIK, Braine L'Alleud (BE)

(73) Assignee: RENAL CARE & RESEARCH, Walhain (BE)

(21) Appl. No.: 18/856,801

(22) PCT Filed: Dec. 29, 2022
(86) PCT No.: PCT/EP2022/088014

§ 371 Date: Oct. 14, 2024

(30) Foreign Application Priority Data
Apr. 14, 2022 (EP) 22168352.7

(51) Int. Cl. G16H 20/10; G16H 20/60; G16H 50/20

(52) U.S. Cl. CPC G16H 20/10; G16H 20/60; G16H 50/20

(57) ABSTRACT

A system suitable for remotely monitoring and maintaining improved hydration of a user, in particular a user suffering from a mineral metabolism disorder. The system comprises a server, a user device in communication with the server, a processing unit operating on the user device, wherein the processing unit is configured to acquire the user's urine parameters, and a care provider device in communication with the server. The system further comprises a prescription unit configured to generate a prescription comprising a recommendation relating to hydration fluid intake by the user, the prescription being based on the user's urine parameters.

### FIELD OF THE INVENTION

The invention relates to the field of hydration fluid prescribing systems. The invention also relates to a method for remotely monitoring a user's hydration state. More specifically, the invention relates to the field of human body hydration optimization methods by using personalized fluid intake prescribing systems and hydration efficacy estimation system by instant remote monitoring of a user's hydration state assessed by urinary parameters and water intake. The system has been designed to maintain an optimal water intake and most appropriate status of hydration (euvolemia) according to the user physical characteristics and associated diseases adapted for telehealth program.

### DESCRIPTION OF PRIOR ART

The importance of adequate fluid intake, mainly water as the best fluid and hydration status in

human health is not longer to be demonstrated. With the increase of human health consciousness, the need of keeping the hydration state of an individual in a physiological desired hydration range has been recognized as critical. Poor fluid intake leads to body underhydration and is a risk factor for arterial hypertension, cardiovascular disease (CVD), chronic kidney disease (CKD) and nephrolithiasis (kidney stones) at the population level. On the contrary high fluid intake can induce body overhydration that can manifest by blood composition abnormalities, swelling or event pulmonary oedema. Those complications are mostly reported in patients with impaired heart or kidney function. Monitoring of water intake and evaluation of body hydration status is increasingly recognized in the management of patients with heart diseases, CKD patients in all stages especially those with advanced stage of kidney failure requiring extra-renal epuration (hemodialysis, peritoneal dialysis) or transplantation, pregnant and breastfeeding women as well as geriatric and pediatric patients. The system monitoring daily fluid intake and evaluating concomitantly body hydration status are necessary to improve body water balance and maintaining the euvolemia. In that context, various systems and methods for human hydration monitoring have been described. Examples of such methods and systems are disclosed for example in US2020/0375533 or in US2008/0195061.

In order to ensure an autonomous usage, most of the known methods and systems for keeping a human body well hydrated are by design meant to be used without any intervention from a care provider. Accordingly, their medical applications, in particular in the context of a telemedicine approach, has been limited so far.

US 2021/074390 A1 proposes a system and a method for sensing properties of a urine sample of a patient. For that, the system comprises a urinalysis device that is mounted on a toilet and in electronic communication with a server, see FIG. 1 for instance. The urinalysis device needs to be calibrated before the measurements.

U.S. Pat. No. 10,928,325 B1 proposes a system that makes nutritional recommendations based on results of an urine test. For that, the user has to apply a urine sample to a card containing multiple tests, and capture an image of the card using a phone. An analysis system executing on the phone or in the cloud may analyze the image and determine test results.

These last two systems enable measuring properties of a urine sample. And based on the results, recommendation of consumption of specific quantities of nutrients for instance can be provided. However, both systems require some specific hardware components: an urinalysis device that is mounted on a toilet and in electronic communication with a server in the first case, and a card containing multiple tests to which a sample urine is applied in the second invention.

### SUMMARY OF THE INVENTION

It is an object of the invention to provide a system suitable for remotely monitoring and improving the hydration body state of a user, for example of a user suffering from a mineral metabolism disorder, and that is simpler with respect to known systems. The invention is defined by the independent claims. The dependent claims define advantageous embodiments. According to the invention, there is provided a system comprising:
a) a server able to operate over a data network, and optionally able to communicated with a database;
b) a user device in communication with the server, preferably via the data network and comprising a user interface for allowing a user to enter one or more urine parameters of said user;
c) a processing unit operating on the user device, wherein the processing unit is configured to acquire said urine parameters about said user when so instructed by the user, and to transmit said urine parameters to the server when so instructed by the user;
d) a care provider device in communication with the server, preferably via the data network, and configured to obtain said user's urine parameters from the server;
   the system further comprising a prescription unit configured to generate and to transmit to the user device a prescription comprising a recommendation relating to hydration fluid intake by the user, the prescription being based on the user's urine parameters transmitted by the user device.
   In the context of the present invention the term “communication” is to be understood as “data communication” and may be a wired or a wireless data communication or a mix of both.

With the system of the invention, a care provider (for example a nephrologist, urologist, clinician, or dietician) can remotely monitor the hydration state of a user or patient, take appropriate steps to improve the hydration state of the user and/or monitor that a pre-established treatment plan or hydration plan is properly adhered to by the user. Further, the system of the present invention is particularly suitable for a telemedicine methodology, enabling specific activities such as telemonitoring, telehealth, telecare or teleconsultation.

In the context of the present invention, it was determined that a correlation exists between increased hydration fluid intake and reduction of a risk of a mineral metabolism disorder, such as the kidney stones recurrence or kidney function decline for example. Indeed, higher fluid intake and better hydration lead to increased glomerular filtration rate resulting in higher production of urine and therefore reduce the propensity of the formation of crystals which could lead to the formation of kidney stones for example.

To the applicant's knowledge, the system of the prevent invention is the first system capable of remotely monitoring and improving hydration of a user based on user data relating to the user's urine parameters. Such parameters are believed to be more accurate and meaningful than other conventional biological parameters of a user, such as for example age, body weight, body mass index, height, for the specific purpose of making a recommendation relating to water intake by the user. The urine parameters have been found to give a more accurate indication of a user's hydration state.

According to the invention, one or more urine parameters need to be entered by the user through

the user interface of the user device. Based on these one or more urine parameters, different prescriptions are possible. Different examples of urine parameters and prescriptions are presented in the detailed description of possible embodiments. For instance, the following urine parameters can be used: volume of urine per 24 hours, urine gravity (or urine specific gravity, or urinary specific gravity (SG); all these terms are known by one skilled in the art), color of urine, and pH value of various urine samples (fasting urine, urine sample after dinner, 24 h urine collection, . . . ) representatives of variable metabolic status (fasting, after the meal intake, or less specific global metabolic state appreciation). Other urine parameters could be used, and the urine parameters can be used in combination. Depending on the values entered by the user for these parameters, different prescriptions are possible. For example, a prescription could be to drink more water per day if one or several urine parameters are outside a range of values. On the contrary, the prescription could be to not change the drinking habits if one or all urine parameters lie in some predetermined optimal ranges. Another example of prescription is to take a specific therapeutic substance or to take more of such a therapeutic substance if one or more urine parameters are outside a targeted range of values.

In the system of the invention, no specific intermediate hardware is used for communicating some data relating to the urine parameters of a user, contrary to the systems disclosed in US 2021/074390 A1 and U.S. Pat. No. 10,928,325 B1. It is rather proposed to provide a user interface on a user device (for instance a mobile phone or smart phone, tablets or personal computer) for allowing the user to enter one or more urine parameters. These urine parameters are determined by the user and communicated by him directly thanks to the user interface. Depending on the type of urine's parameter, different determination procedures exist. If the urine parameter is a color of the urine, then it is preferably determined directly by the user, possibly by comparing the color of a urine sample with a reference scale, provided preferably directly on the user interface. If the urine parameter is a pH (or pH value), then such a pH value is preferably determined by the user with the help of a pH measuring stick known by one skilled in the art. But, finally the user enters herself/himself one or more urine parameter about him, thanks to the user interface of the user device. There is no additional intermediate hardware such as the urinalysis device mounted on a toilet or a card containing multiple tests, from which an image is captured. The user determines herself/himself the value that she/he uploads on the user device directly without any supplementary steps or hardware or manipulation (image analysis for instance). The system of the invention is thus simpler, more flexible, and more user friendly, applicable everywhere, every time when a user urinates. That solution is different from those disclosed in US 2021/074390 A1 (requiring the access for the toilets equipped by a specific hardware) and U.S. Pat. No. 10,928,325 B1 (system requiring specific cards and specific software for image analysis to provide the data). Moreover, the user device can be a personal mobile phone on which a dedicated application has been uploaded and installed, in order to perform the method of the invention. The user interface allows a user to enter urine parameters about her/him and that directly. Preferably, that is done manually or via some voice recognition. One can thus gather more information about urine parameters, and from more people (the system of the invention can accompany a patient or user everywhere) contributing to the increase in the reliability between urine parameters and lifestyle. This leads to a better understanding of user fluid intake behaviors and allows significantly more efficient follow-up of patients at lower cost (no specific hardware needed with the system of the invention).

Hydration status is evaluated by Clinician or dialyze monitor in US 2021/074390 A1. Patients do not have any information about the degree of hydration and any information about 24 h urine volume contrary to the system of the invention. In the system of US 2021/074390, data is recorded but there is no instant feedback loop that provides to the patient information about the hydration quality or action about hydration to take. The system presented in US 2021/074390 serves for programming the software of dialyse monitor for further blood treatment and not for fluid intake or therapeutic substance prescription directly. The system of US 2021/074390 A1 does not provide any prescription of personalize water intake with a formula ml/Kg and water intake plan, and any instant adaptation in correlation with uploaded urine data. US 2021/074390 A1 does not teach to use urine color for providing a prescription. The system of US 2021/074390 A1 can only be used in toilettes where the device is available. The system of the invention can be used everywhere. US 2021/074390 A1 does not teach to use a database.

In an advantageous embodiment of the system according to the invention, the prescription unit operates on the care provider device. This embodiment is beneficial in many aspects as it allows continuous monitoring of the hydration state of an individual and continuous and preferably real-time communication and care delivery between the user and the (health) care provider. US 2021/074390 A1does not allow a real time communication.

In another advantageous embodiment of the system, the user's urine parameters for use herein are selected from the group consisting of urine volume, urine pH, urinary specific gravity (or urine specific gravity (SG)), urine color, urine biomarkers of kidney injury, infection, stress or inflammation and any combinations thereof. These parameters have been found to constitute the most meaningful parameters for enabling remote monitoring and maintaining optimized hydration of a user, and therefore providing an improved recommendation relating to hydration fluid intake by the user. According to a possible embodiment, only urine colour is entered by the user. According to another possible embodiment, only pH is entered by the user. According to yet another possible embodiment, only urinary specific gravity is entered by the user.

According to another advantageous embodiment, the system as described herein further comprises an alarm unit configured to provide an alert, for example an alert to the user via the user device and/or an alert to the care provider via the care provider device, when one or more of the following user's urine parameters conditions are met:
a) the daily urine volume is lower than 3.0 L/24 h, or lower than 2.8 L/24 h, or lower than 2.6 L/24 h, or lower than 2.5 L/24 h, or lower than 2.4 L/24 h, or lower than 2.2 L/24 h, or lower than 2.0 L/24 h;
b) the urinary specific gravity higher than 1.010, or higher than 1.020, or higher than 1.030;
c) the urine pH is outside the range from 5.0 to 7.0, or outside the range from 5.5 to 6.5;
d) the urine colour scale is higher than 3 according to the Armstrong visual scale (which ranges from 1 to 8). This Armstrong scale is well-known by one skilled in the art.
   These urine parameters conditions have been found to constitute the most meaningful and critical conditions to monitor when it comes to triggering a proper recommendation relating to hydration fluid intake by the user, in particular a recommendation to increase a hydration fluid intake.
   According to a particular embodiment, the alarm unit for use in the system of the present invention is advantageously selected from the group consisting of visual alarms, audible alarms, sensory alarms, and any combinations thereof. These particular forms of alarm units have been found to provide the most efficient alerts to raise the attention of the user and/or of the care provider, and thus enable a more responsive and dynamic adjustment of the prescription due to changing conditions. This in turn, enables a more efficient control of the adherence to a pre-established treatment plan or to an adjusted treatment plan. US 2021/074390 A1 does not provide any alert unit.

In some embodiments of the system according to the invention, the prescription generated by the prescription unit further comprises a recommendation relating to the intake of a therapeutic substance by the user. This specific embodiment is particularly beneficial as it provides the care provider an additional tool to personalize and to more quickly adjust the overall recommendation by advantageously complementing the hydration fluid intake recommendation. In a more advantageous embodiment, the therapeutic substance for use herein takes the form of a pill and is beneficially selected from the group of alkalinizing agents. More advantageously, the therapeutic substance is selected from the group consisting of sodium bicarbonate, potassium and/or magnesium citrate, lanthanum and/or calcium carbonate, calcium acetate, oxalate degrading enzymes, and any mixtures thereof. These specific therapeutic substances are particularly suitable to adjust, more in particular to increase, the urine pH of the user in case the treatment plan requires so. In a still preferred embodiment, the recommendation relating to the intake of a therapeutic substance by the user is based on the user's urine parameters transmitted by the user device.

According to some embodiments of the system of the invention, the prescription generated by the prescription unit is communicated from the care provider device to the user device, in particular via the data network. This embodiment is particularly beneficial as it allows communication and care delivery between the user and the (health) care provider, which in turn enables a more efficient and dynamic control of the adherence to the pre-established treatment plan or to an adjusted treatment plan. Further, this particular embodiment is particularly efficient in the context of a telemedicine approach.

According to another advantageous embodiment, the system of the invention further comprises a hydration fluid delivery article in communication with the user device. This embodiment is particularly advantageous as it enables a more convenient adherence by the user to the pre-established treatment plan or to an adjusted treatment plan.

In some advantageous embodiments of the system, the processing unit for use herein is further configured to establish a hydration fluid consumption plan, wherein the hydration fluid consumption plan comprises a plurality of hydration fluid drinking events scheduled at predetermined intervals, wherein each hydration fluid drinking event is associated with a specific hydration fluid intake target, and wherein the processing unit is further configured to compare the hydration fluid intake with the specific hydration fluid intake target for each hydration fluid consumption event and to cause the prescription unit to generate a prescription comprising a recommendation for the user to consume more hydration fluid if the hydration fluid intake during the hydration fluid consumption event is smaller than the specific hydration fluid intake target associated with that hydration fluid consumption event, wherein the prescription is provided before the next hydration fluid consumption event. This particular embodiment is particularly beneficial as it not only allows the care provider to establish a personalized hydration fluid consumption plan for the user, but it further enables both the user and the care provider to ensure that the hydration fluid consumption plan is adhered to in a continuous, dynamic, proactive and autonomous manner.

In some other embodiments of the system according to the invention, the prescription unit for use herein is further configured to generate a revised prescription depending on the adherence of the user to the hydration fluid consumption plan. This embodiment is particularly advantageous as it enables the care provider or the prescription unit itself to dynamically adjust the prescription, in particular the recommendation relating to the hydration fluid and/or the therapeutic substance intake by the user, and make sure that the overall hydration fluid consumption plan is still adhered to even in those cases where the initial recommendation was not respected by the user.

According to a particularly advantageous embodiment, the system of the invention is particularly suitable for remotely advising a user suffering from mineral metabolism disorders, in particular kidney stones (or nephrolithiasis) and heart diseases, CKD patients in all stages especially those with advanced stage of kidney failure requiring extra-renal epuration (hemodialysis, peritoneal dialysis) or transplantation, pregnant and breastfeeding women as well as geriatric and pediatric patients. This embodiment is particularly useful as it allows a care provider to advise and assist a user in managing and/or preventing mineral metabolism disorder conditions.

In one beneficial embodiment, the system of the invention further comprises any of:
a) a learning module which is in particular supported by artificial intelligence, machine learning and deep machine learning protocols and algorithms;
b) an education module;
c) a (self) motivation module;
d) a reward module;
e) a quality-of-life assessment module; and
f) a diet recommendation module.
   According to this specific execution, the system of the invention can further improve its capabilities and the quality of the prescription generated by the prescription unit. According to this particular execution still, the user is enabled to take a more proactive and rewarding part in the overall treatment plan, which in turns contribute to providing a more satisfying user experience.

According to another possible embodiment, the system of the invention further comprises an additional connected device for monitoring some physiological parameters of the user. That additional connected device is preferably able to communicate with the server, preferably via the data network. According to a preferred embodiment, it is also able to communicate with the user device. Preferably, the care provider device is configured to obtain data provided by said additional connected device.

That additional connected device preferably allows monitoring blood pressure and/or glycemia and/or body composition. However, other physiological parameters may be monitored by such additional connected device. According to a possible example, the additional connected device is able to measure parameters of inflammation and/or kidney injury, urine biomarkers of kidney injury, infection, stress or inflammation for instance in a urine sample. That is particularly interesting for patients suffering from cancer, acute or chronic graft rejection or nephrotoxicity of drugs.

Preferably, the prescription comprising a recommendation relating to hydration fluid intake by the user is based on data monitored and transmitted by the additional connected device. This allows making a link between the recommendation of drinking water typically and the physiological parameters monitored by the additional connected device. This is particularly useful and interesting in a telemedicine approach.

It is another object of the invention to provide a method for remotely monitoring a user's hydration state, comprising the steps of:
a) providing a system as described above;
b) prompting the user, from the user interface of the user device, to enter urine parameters about him;
c) receiving said urine parameters;
d) operating the prescription unit to generate a prescription comprising a recommendation relating to hydration fluid intake by the user, and wherein the prescription is based on the user's urine parameters entered by the user on the user device.
   According to the method of the invention, the urine parameters are entered directly by the user to the user device, through the user interface. So, without the use of a urinalysis device mounted on a toilet or a card containing multiple tests for instance. Preferably, the urine parameters are entered manually by the user to the user device, through the user interface. In another possible embodiment, the urine parameters are entered through a voice recognition software module. According to the invention, the urine parameters that are entered to the user device are determined by the user or patient. For some urine parameters such as the urine pH, a pH measuring stick is used but it is the user who determines the pH value to enter to the user device, typically from the color or the pH measuring stick exposed to urine. The same situation occurs with other urine parameters such as color or gravity (or density) of the urine. The related advantage is to have a system and method that is more simple to use for all. It is also cheaper than existing solutions as no intermediate measuring device such as urinalysis device mounted on a toilet or a card containing multiple tests is necessary.

According to an advantageous embodiment, the method of the invention is particularly suitable for remotely advising a user suffering from mineral metabolism disorders, in particular kidney stones (or nephrolithiasis) and heart diseases, CKD patients in all stages especially those with advanced stage of kidney failure particularly if they undergo extra-renal epuration (hemodialysis, peritoneal dialysis) or transplantation, pregnant and breastfeeding women as well as geriatric and pediatric patients.

It is yet another object of the invention to provide a computer program for causing a computer to execute a step of generating a prescription comprising a recommendation relating to hydration fluid intake which is suitable for a user, and wherein the prescription is based on data relating to the user's urine parameters.

In an advantageous embodiment of the program according to the invention, the prescription further comprises a recommendation relating to the intake of a therapeutic substance suitable for the user.

### SHORT DESCRIPTION OF THE DRAWINGS

These and further aspects of the invention will be explained in greater detail by way of examples and with reference to the accompanying drawings in which:

FIG. 1 shows a diagram depicting a schematic configuration of a system according to an exemplary embodiment of the present invention;

FIG. 2 shows a diagram depicting a schematic configuration of a user device, a processing unit and a hydration fluid delivery article for use in a system according to one exemplary embodiment of the present invention;

FIG. 3 shows a block diagram depicting a schematic configuration of a server for use in a system according to one exemplary embodiment of the present invention;

FIG. 4 shows a block diagram schematically depicting a possible embodiment of the method of the invention;

FIG. 5 shows a color scale that can be used for determining a color of a urine sample;

FIG. 6 shows same color scale in grey levels.

The drawings of the figures are neither drawn to scale nor proportioned. Generally, similar or identical components are denoted by the same reference numerals in the figures. The accompanying figures are intended to provide a better understanding of some embodiments. In particular, the figures should not be used for reducing the scope of the invention.

### DETAILED DESCRIPTION OF POSSIBLE EMBODIMENTS

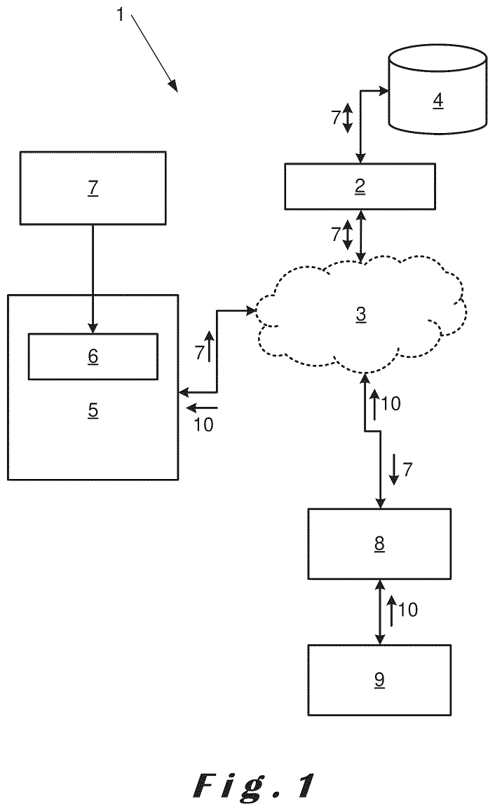

In reference to FIG. 1, there is provided a system 1 comprising:
a) a server 2 able to operate over a data network 3, and optionally connected to a database 4;
b) a user device 5 in communication with the server 2, preferably via the data network 3, and comprising a user interface for allowing a user to enter one or more urine parameters about him;
c) a processing unit 6 operating on the user device 5, and configured to acquire said urine parameters when so instructed by the user, and to transmit said urine parameters to the server 2 when so instructed by the user; according to a possible embodiment, the acquisition and the transmission of the urine parameters 7 to the server 2 are performed simultaneously;
d) a care provider device 8 in communication with the server 2, preferably via the data network 3 and configured to obtain the urine parameters of said user data from the server 2. The system 1 further comprises a prescription unit 9 configured to generate and to transmit to the user device 5 a prescription 10 comprising a recommendation relating to hydration fluid intake by the user, and wherein the prescription 10 is based on the user's urine parameters transmitted by the user device 5.

As will be apparent to those skilled in the art, a server 2 for use herein may perform various typical operations and functions. Exemplary operations include processing and analysing various data such as user's personal data and external data, requesting input, providing instructions, sending messages and alerts. The server 2 for use herein may be assisted with suitable computer programs, software's, algorithms, machine learning protocols and models, and may in some examples also be provided with artificial intelligence capabilities. Suitable servers 2 for use herein are not particularly limited and will be easily identified by those skilled in the art in the light of the present disclosure. The data networks 3 for use herein are not particularly limited. An exemplary suitable data network 3 is the Internet network. Similarly, databases 4 for use herein will be easily identified by those skilled in the art and are not particularly limited either. Databases 4 for use herein are typically provided with suitable memory or data storage units which are typically able to store various types of data, in particular the user data relating to the user's urine parameters 7 and data relating to the prescription 10, and/or the hydration fluid intake by the user, and/or the therapeutic substance intake by the user, and/or the adherence of the user to the hydration fluid consumption plan, and/or the therapeutic substance intake plan.

The system 1 of the invention further comprises a user device 5 in communication with the server 2, preferably via the data network 3. A suitable user device 5 for use herein is for example a so-called terminal device, which usually comprises appropriate data communication means to ensure proper connectivity with a data network 3 and access to the server 2. In some embodiments, the user device 5 may comprise memory or data storage units that are suitable to store various data, such as in particular user's personal data and data relating to the user's urine parameters 7. In some other embodiments, the user device 5 may comprise an interactive display or interface.

Suitable user devices 5 for use herein include, but are not limited to, smart mobile devices. Suitable smart mobile devices comprise smartphones, tablets, laptops, or any other handheld or portable connected devices. In an advantageous embodiment, the user device 5 is a smartphone. The system 1 of the invention further comprises a processing unit 6 operating on the user device 5, configured to acquire and process the user data relating to the user's urine parameters 7. Processing units 6 for use herein will be easily identified by those skilled in the art. A suitable processing unit 6 is for example a microcomputer. In an advantageous embodiment, the user device 5 is associated with a suitable computer program or software application. Suitable applications are for example (integrated) mobile applications or web applications running on remote or mobile web browsers. According to the present invention, the processing unit 6 preferably operates on the user device 5. However, it is not necessarily located and integrated into the user device 5. In some embodiments, the processing unit 6 may be located in the server 2, or in any other suitable parts of the system 1. In an advantageous embodiment, the user device 5 for use herein is configured to receive, store, process and communicate user data relating to the user's urine parameters 7 and/or to hydration fluid intake by the user. The system 1 of the invention further comprises a care provider device 8 in communication with the server 2, preferably via the data network 3. Similar to the user device 5, a suitable care provider device 8 for use herein is for example a so-called terminal device, which usually comprises appropriate data communication or transmission means to ensure proper connectivity with the data network 3 and access to the server 2. In some embodiments, the care provider device 8 may comprise memory or data storage units that are suitable to receive and store various data, such as for example user's personal data and the user data relating to the user's urine parameters 7. In some other embodiments, the care provider device 8 may comprise an interactive display or interface.

Suitable care provider devices 8 for use herein include, but are not limited to, personal computing devices or smart mobile devices. Suitable smart mobile devices comprise smartphones, tablet, laptops, or any other handheld or portable connected devices. In an advantageous embodiment, the user device 5 is a personal computing device, such as a desktop or laptop computer for example.

In an advantageous embodiment, the care provider device 8 for use in the system 1 of the invention is associated with a suitable computer program or software application. Suitable applications are for example (integrated) desktop applications or web applications running on remote or mobile web browsers. In a typical embodiment, a computing unit operates on the care provider device 8. The computing unit for use herein is not necessarily located and integrated into the care provider device 8. In some embodiments, the computing unit operating on the care provider device 8 may be located in the server 2, or in any other suitable parts of the system 1. In an advantageous embodiment, the care provider device 8 for use herein is configured to receive, store, process and communicate user data relating to the user's urine parameters 7 and/or to hydration fluid intake by the user. In a particularly advantageous embodiment, the care provider device 8 is provided with a visual dashboard.

The system 1 of the invention further comprises a prescription unit 9 configured to generate a prescription 10. Prescription units 9 for use herein will be easily identified by those skilled in the art. A suitable prescription unit 9 is typically a microcomputer. In a preferred embodiment, the prescription unit 9 operates on the care provider device 8. The prescription unit 9 for use herein is not necessarily located and integrated into the care provider device 8. In some embodiments, the prescription unit 9 may be located on the server 2, or in any other suitable parts of the system 1. In an advantageous embodiment, the prescription unit 9 for use herein is configured to receive, store, process and communicate user data relating to the user's urine parameters 7 and/or to the hydration fluid intake by the user. According to an exemplary embodiment, the prescription unit 9 for use herein is associated or in communication with the computing unit operating on the care provider device 8. In a possible embodiment, the prescription unit 9 operates on the care provider device 8, and the care provider device 8 is in communication with the server 2 via the data network 3. Accordingly, the prescription 10 generated by the prescription unit 9 is for example communicated by the care provider device 8 to the user device 5, in particular via the data network 3. In some examples, the user's urine parameters 7 are selected from the group consisting of urine volume, urine pH, urinary specific gravity, urine colour, content of additional urine (biochemical) components, and any combinations thereof. Additional urine (biochemical) components comprise, but are not limited to, red cells, white cells, leucocytes, bacteria, urobilinogen, creatinine, urea, uric acid, sodium, chlore, potassium, calcium, oxalates, phosphate oxalate, citrates, nitrates, sulphates, glucose, proteins, and any combinations or mixtures thereof.

In an exemplary embodiment, the prescription unit 9 generates a personalized prescription 10 comprising a recommendation relating to hydration fluid intake by the user to ensure an improved hydration level for the user, wherein the prescription 10 is repeatedly adapted in function of the user data relating to the user's urine parameters 7, and wherein those user data are received from a remote location, typically from the user device 5. As such, the system of the present invention may be considered as a hydration fluid prescribing system, as the prescription 10 comprises a recommendation relating to hydration fluid intake by the user. The disclosed system 1 may further be referred to as a system suitable for remotely monitoring a user's hydration state and/or a user's urinary parameters 7. The system as described herein may further be regarded as system suitable for (re) alkalinisation of a user's urine.

According to an advantageous embodiment, the system 1 of the invention further comprises an alarm unit (not shown) configured to provide an alert to the user and/or to the care provider when one or more of the following user's urine parameters (unfavorable) conditions are met:
a) the daily urine volume is lower than 3.0 L/24 h, or lower than 2.8 L/24 h, or lower than 2.6 L/24 h, or lower than 2.5 L/24 h, or lower than 2.4 L/24 h, or lower than 2.2 L/24 h, or lower than 2.0 L/24 h;
b) the urinary specific gravity is higher than 1.010, or higher than 1.020, or higher than 1.030;
c) the urine pH is outside the range from 5.0 to 8.0, or outside the range from 5.5 to 6.5;
d) the urine colour scale is higher than 3 according to the Armstrong visual scale (which ranges from 1 to 8).

Alarm units configured to provide an alert and for use in the present system 1 are not limited. Exemplary alarm units include visual alarms, audible alarms, sensory alarms, and any combinations thereof. Alarm units for use herein may be located in any suitable parts of the system 1.

In an advantageous embodiment, the alarm unit is located in any of the user device 5, the care provider device 8, and any combinations thereof. More advantageously, the alarm unit for use herein is located or is at least partly comprised in the user device 5. Typically, in those situations where the alert is provided due one or more of the above-detailed unfavourable user's urine parameters conditions being met, this would trigger a recommendation relating to hydration fluid intake, in particular a prompt to the user to increase his hydration fluid intake.

In one particularly advantageous embodiment of the system 1 of the invention, the prescription 10 further comprises a recommendation relating to the intake of a therapeutic substance by the user. The therapeutic substance for use herein is typically meant to contribute to adjusting or modifying the user's urine parameters 7 so that they fall within a predetermined range or to get close to a predetermined value. More particularly, the therapeutic substance is designed for none of the above-detailed user's unfavourable urine parameters conditions to be met, or none to be met anymore, after ingestion of the therapeutic substance by the user. Even more advantageously, the therapeutic substance is designed for the none of the above-detailed unfavorable user's urine parameters 7 conditions to be met in conjunction with the hydration fluid intake.

The therapeutic substance is for example a substance which may take the form of a pill, a capsule, a tablet, a bead, drops of a liquid substance, or any combinations or mixtures thereof.

Preferably, the therapeutic substance for use herein is selected from the group consisting of alkalinizing agents, oxalate binding agents, oxalate degrading agents, phosphate binding agents, potassium binding agents, and any combinations or mixtures thereof. More preferably, the therapeutic substance is selected from the group consisting of potassium citrate, magnesium citrate, sodium bicarbonate, lanthanum carbonate, calcium carbonate, any aluminum or iron based preparation, sevelamer based preparations, sodium zirconium cyclosilicate, patiromer sorbited calcium, sucroferric oxyhydroxide, calcium acetate, reloxaliase, myo-inositol hexakisphosphate (IP-6) analogue, pH-tolerant oxalate degrading enzymes (such as e.g. Oxidien: OX-1), oxalate-degrading enzymes, rationally designed microbial consortia, genetically engineering microbial medicines, lumasiran and any combinations or mixtures thereof.

More preferably, the therapeutic substance for use herein is selected from the group of alkalinizing agents, more in particular the therapeutic substance is selected from the group consisting of sodium bicarbonate, potassium and/or magnesium citrate, lanthanum and/or calcium carbonate, calcium acetate, oxalate-degrading enzymes, and any combinations or mixtures thereof.

According to the embodiment of the system 1 where the prescription 10 further comprises a recommendation relating to the intake of a therapeutic substance, any of the user device 5, the care provider device 8, the prescription unit 9, the processing unit 6 or the computing unit are further configured to receive, store, process and communicate data relating to the intake of the therapeutic substance.

In an advantageous embodiment of the system 1 of the invention, the prescription unit 9 for use herein is further configured to generate and communicate, preferably in real-time, a revised prescription 10 based on data relating to any of the therapeutic substance intake or hydration fluid intake.

In one particular embodiment of the system 1, the prescription 10 generated by the prescription unit 9 is communicated by the care provider device 8 to the user device 5, preferably via the data network 3.

In another beneficial embodiment, the system 1 of the invention further comprises a hydration fluid delivery article 11 which is in communication with the user device 5.

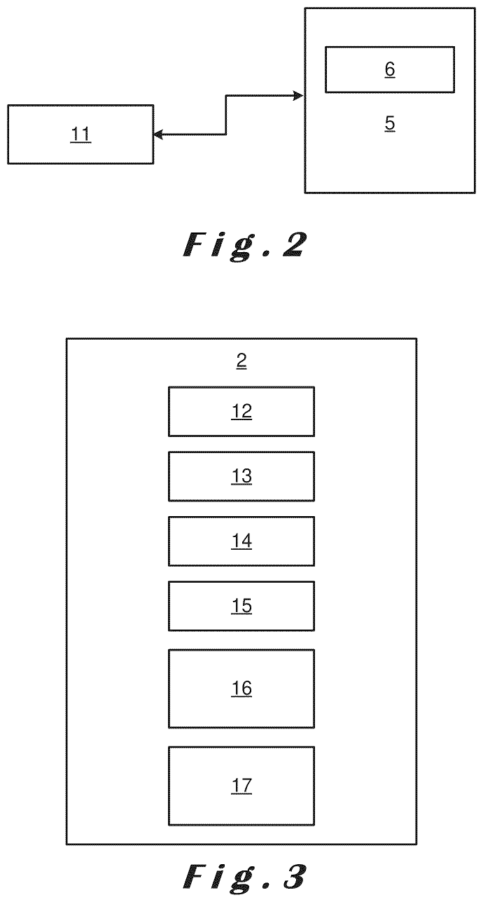

FIG. 2 shows a diagram depicting a schematic configuration of a user device 5 comprising a built-in processing unit 6 in communication with an exemplary hydration fluid delivery article 11 for use in the system 1 of the present invention. Different types of hydration fluid delivery articles 11 can be used. Exemplary hydration fluid delivery articles 11 include containers, cans, bottles, canisters or boxes.

According to another beneficial embodiment of the system 1, the processing unit 6 is further configured to establish a hydration fluid consumption plan, wherein the hydration fluid consumption plan comprises a plurality of hydration fluid drinking events scheduled at predetermined intervals, wherein each hydration fluid drinking event is associated with a specific hydration fluid intake target, and wherein the processing unit 9 is further configured to (continuously) compare the hydration fluid intake with the specific hydration fluid intake target for each hydration fluid consumption event and to cause the prescription unit 6 to generate and communicate a prescription 10 comprising a recommendation-for example a visual indication-for the user to consume more hydration fluid if the hydration fluid intake during the hydration fluid consumption event is smaller than the specific hydration fluid intake target associated with that hydration fluid consumption event, wherein the prescription 10 is provided before the next hydration fluid consumption event.

Advantageously, the prescription unit 9 for use herein is further configured to generate (and communicate), preferably in real-time, a revised prescription 10 depending on the adherence of the user to the hydration fluid consumption plan, wherein the revised prescription is for example based on data relating to any of the therapeutic substance intake or hydration fluid intake by the user. Advantageously still, the system 1 of the present invention may further comprise any of:
a) a learning module 12 which is in particular supported by artificial intelligence-based protocols and algorithms, more in particular deep machine learning;
b) an education module 13;
c) a (self) motivation module 14;
d) a reward module 15;
e) a quality-of-life assessment module 16; and
f) a diet recommendation module 17, in particular a recommendation module relating to low oxalate and low calcium diet.
   The various additional modules for use herein may be located in any suitable parts of the system 1. Advantageously, the additional modules are located on the server 2. FIG. 3 shows a block diagram depicting a schematic configuration of a server 2 for use in a system 1 according to the present invention, wherein said server 2 comprises a learning module 12, an education module 13, a (self) motivation module 14, a reward module 15, a quality-of-life assessment module 16, and a diet recommendation module 17. The system 1 of the present invention is particularly suitable for remotely monitoring and/or advising a user suffering from mineral metabolism disorders, in particular kidney stones (or nephrolithiasis) and chronic kidney diseases (from stage 1 to stage 5). The system 1 of the present invention is also particularly suitable for remotely monitoring and/or advising a user suffering from dehydration. The system 1 is further suitable for remotely monitoring and/or advising a user subject to dialysis and/or transplantation, and/or suffering from a heart disease.

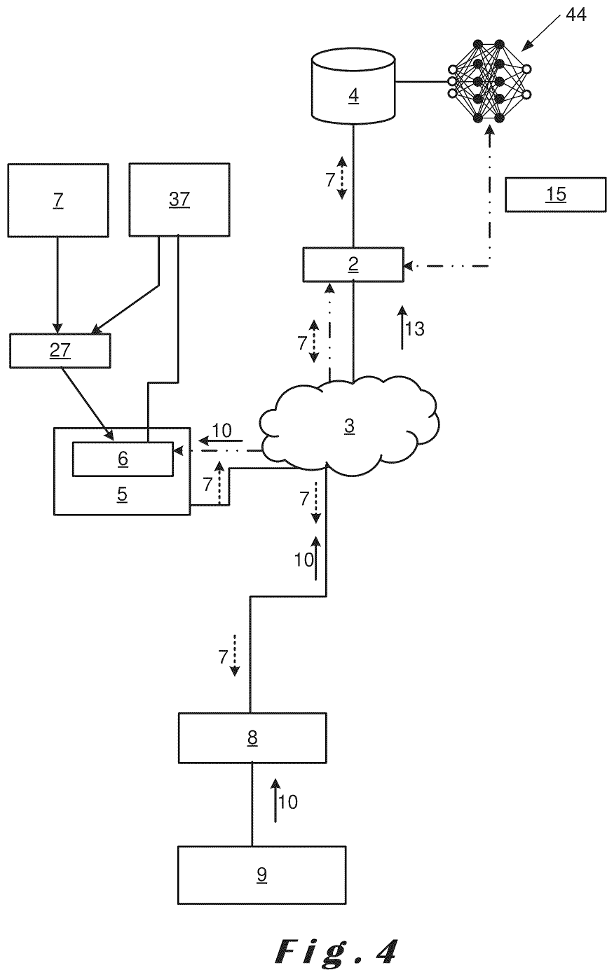

FIG. 4 schematically presents a possible embodiment of the system of the invention and the related method. As explained before, urine parameters 7 are entered by the user through a user interface of a user device 5. Preferably, this is done manually 27. Preferably also, there is an additional connected device 37 for monitoring some biological and/or physiological parameters of the patient/user. Data provided by said additional connected device 37 can be entered to the user device 5 manually 27 or through a communication, for instance a wifi or Bluetooth standard. Based on these urine parameters 7 and preferably also based on the data provided by the additional connected device 37, a prescription 10 is provided to the user. Preferably, a neural network module 44 and/or a deep machine learning module is used in order to generate the prescription 10 based on these urine parameters 7. As shown in FIG. 4, an education module 13 and/or a reward module 15 can also be used.

The inventors propose different possible prescriptions 10 based on urine parameters 7 of the user. In a same way, different examples of urine parameters 7 are possible. According to a first example, only one urine parameter 7 is used and entered by the user. An example of such a single urine parameter 7 to use is a volume of urine of the user. For instance, and for a given user, a user or patient measures a volume of urine larger than 2.5 l in 24 h, and enters it thanks to the interface of the user device 5. For such a value of that urine parameter 7, the prescription unit 9 preferably transmits a prescription 10 with a recommendation of no change in terms of water intake. If the user enters a volume of urine smaller than 2.5 l in 24 h, then the prescription unit 9 preferably transmits a prescription 10 with a recommendation of increasing the water intake. For instance, the prescription 10 may comprise a recommendation of increase of water intake by personalized quantity of fluid in ml per day (for instance increase of 250 ml per day). Preferably, the prescription 10 further comprises a recommendation of recontroling the volume of urine one day after, and to repeat the control of the volume of urine until the volume of urine per 24 hours is larger than 2.5 l.

The system 1 preferably uses neuron networks and artificial intelligence to improve the adherence

and to consume water volume to drink accordingly to the formula of X ml/kg of body weight/day. With the system 1 of the invention, the instant adaptation the amount of water to drink according to the circumstances of the environment, the effort, and the working conditions, . . . , is possible. The system 1 defines the amount of water to consume to optimize hydration by specialized theoretical education (push notification and explanation the issue for kidney stones prevention) and by assistance in the process of therapeutic education and reinforcement of adherence applying the behavior correction preferably by using positive neurofeedback reinforcement techniques (push notification). If a patient achieves the goal defined according to the patient's characteristics by dashboard user such as: urine volume >2,5 L/24, the patient will receive the notification of congratulation. The feedback may be provided every day and sent to a dashboard; by time-interval according to the patient adherence to perform 24 h urine collection and record the requested value. Instantaneous calculation of volume water per slot per time period: 200-250ml every 2 hours or adapted according to the patient activities. The system 1 may adapt the water intake in case of exercise intensity: Low: 1000 mL; High: 2000 mL and offer the opportunities to choose the times when patients choose to drink as well as depending on the location and time of the work pattern. If the environment is hot, a recommendation could be as follow: ‘Increase 500-1000 mL and check the specific gravity of the urine and pH to ensure the recommended volume is correct’. If unable to drink during the day (taxi drivers for instance) the system 1 may adapt alerts or adjust the volume and hydration hours accordingly to the patient choice.

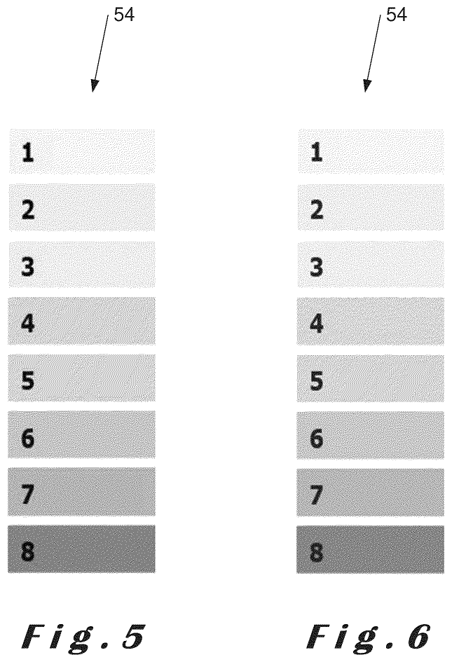

According to another example, urine color is a urine parameter 7 that is entered by the user through the interface of the user device 5, and used by the system 1 for generating a prescription 10. FIG. 5 shows an example of color scale 54 that could be used. FIG. 6 is an equivalent of FIG. 5, but in a greyscale format (in practice, only the scale in color of FIG. 5 is useful). Preferably, the color scale 54 used to determine the color of the urine is an Armstrong scale. Then, the user uses such a color scale 54 for determining where lies his/her own urine sample in terms of colors. In the example shown in FIG. 5 (of FIG. 6), if the urine color is lower than 4, then the prescription 10 preferably comprises a recommendation of not changing the hydration fluid intake. If the urine color is higher than score 3 in the scale of FIG. 5 (or FIG. 6), then the prescription 10 preferably comprises a recommendation of increasing the hydration fluid intake, for instance a water intake. Preferably, it is recommended to increase water intake by personalized quantity of fluid in ml per day, and to recontrol urine color the day after. Still preferably, it is recommended to repeat the control of color until the urine color is lower than 4.

According to another example, urinary specific gravity is a urine parameter 7 that is entered by the user through the interface of the user device 5, and used by the system 1 for generating a prescription 10. Preferably, if urinary specific gravity is lower than or equal to 1.010, then the prescription 10 preferably comprises a recommendation of not changing the hydration fluid intake. Preferably, if urinary specific gravity is higher than 1.010, then the prescription 10 preferably comprises a recommendation of drinking some water. And preferably to increase water intake by personalized quantity of fluid in ml per day. Preferably, the system 1 further recommends in that case to recontrol the urinary specific gravity one day after, and still preferably to repeat the control until the urinary specific gravity is lower than or equal to 1.010.

According to another example, pH of urine is a urine parameter 7 that is entered by the user through the interface of the user device 5, and used by the system 1 for generating a prescription 10. Assuming that the user follows an alkalizing treatment, if a pH value is within the range 6.0 to 6.5, then the prescription 10 preferably comprises a recommendation of not changing the treatment. If a pH value lower than 6.0 is entered by the user, then the prescription 10 preferably comprises a recommendation of increasing the alkalinization treatment, for instance by taking one more pill daily. Still preferably, the system 1 also recommends in that case to recontrol the pH of the urine one day after, until a target of pH value is achieved. If a pH value larger than 6.50 is entered by the user, then the prescription 10 preferably comprises a recommendation of taking one pill less daily. Still preferably, the system 1 also recommends in that case to recontrol the pH of the urine one day after, until a target of pH value is achieved. Preferably, users should have an option to enter the type of method they use to measure pH. For instance, classic dipstick or laboratory strip.

According to a possible embodiment, several urine parameters 7 are entered by the user through the user interface of the user device 5, and the prescription 10 is based on such several urine parameters 7. For instance, the following parameters are used in combination for determining the prescription 10: color, specific gravity, pH value, and volume of urine. For instance if the urine color is lower than 3 in the scale of FIG. 5, gravity is lower than 1.010, pH ranging between 6.0 and 6.5, and a volume of urine per 24 hours is larger than 2.5 l, then the prescription 10 comprises a recommendation of not changing water intake. If one of these urine parameters 7 lies outside these ranges, then the prescription 10 may comprise a recommendation of drinking more water and/or relating to an intake of a therapeutic substance.

It is another object of the invention to provide a method for remotely monitoring a user's hydration state, comprising the steps of:
a) providing a system 1 as described above;
b) prompting the user, from a user interface of the user device 5, to enter urine parameters 7 about him;
c) receiving said urine parameters 7;
d) operating the prescription unit 9 to generate a prescription 10 comprising a recommendation relating to hydration fluid intake by the user, and wherein the prescription 10 is based on the user's urine parameters 7 entered by the user on the user device 5.
   In an advantageous embodiment, the method of the present invention further comprises the steps of:
e) operating the prescription unit 9 for it to establish a personalized hydration fluid consumption plan, wherein the hydration fluid consumption plan comprises a plurality of hydration fluid consumption events scheduled at predetermined intervals, wherein each hydration fluid consumption event is associated with a specific hydration fluid intake target,
f) further operating the prescription unit 9 to compare the hydration fluid intake with the specific hydration fluid intake targets for each hydration fluid consumption event, and
g) further operating the prescription unit 9 to generate and communicate a prescription 10 comprising a recommendation—in particular a visual indication—for the user to consume more hydration fluid if the hydration fluid intake during the hydration fluid consumption event is smaller than the specific hydration fluid intake target associated with that hydration fluid consumption event, wherein the prescription 10 is provided before the next hydration fluid consumption event.

According to an advantageous embodiment, the method of the invention is particularly suitable for remotely advising a user suffering from a mineral metabolism disorder, in particular kidney stones (also called nephrolithiasis) and/or a chronic kidney disease (from stage 1 to stage 5). The method is also suitable for remotely advising a user suffering from dehydration or subject to dialysis and/or being transplanted, as well as for advising a user suffering from a heart disease.

It is yet another object of the invention to provide a computer program for causing a computer to execute a step of generating a prescription comprising a recommendation relating to a hydration fluid intake which is suitable for a user, and wherein the prescription 10 is based on urine parameters 7 of the user.

In an advantageous embodiment of the program according to the invention, the prescription 10 further comprises a recommendation relating to the intake of a therapeutic substance suitable for the user. The computer program according to the invention typically stores executable instructions, which when executed by a computer, cause the computer to execute the step of generating the prescription 10 or the method as described above.

The present invention has been described in terms of specific embodiments, which are illustrative of the invention and not to be construed as limiting. More generally, it will be appreciated by persons skilled in the art that the present invention is not limited by what has been particularly shown and/or described hereinabove. Reference numerals in the claims do not limit their protective scope. Use of the verbs “to comprise”, “to include”, “to be composed of”, or any other variant, as well as their respective conjugations, does not exclude the presence of elements other than those stated. Use of the article “a”, “an” or “the” preceding an element does not exclude the presence of a plurality of such elements.

The invention may also be described as follows: a system 1 suitable for remotely monitoring and maintaining improved hydration of a user, in particular a user suffering from a mineral metabolism disorder. The system 1 of the invention comprises a server 2 operating over a data network 3, and optionally connected to a database 4, a user device 5 in communication with the server 2 via the data network 3, a processing unit 6 operating on the user device 5, wherein the processing unit 6 is configured to acquire user data relating to the user's urine parameters and to transmit said user data to the server 2, and a care provider device 8 in communication with the server 2 via the data network 3 and configured to obtain said user data from the server 2. The system 1 further comprises a prescription unit 9 configured to generate and to transmit to the user device 5 a prescription 10 comprising a recommendation relating to hydration fluid intake by the user, and wherein the prescription 10 is based on the user data relating to the user's urine parameters 7.

1. A system, comprising:
a server operational over a data network, and optionally connected to a database;
a user device configured to communicate with the server via the data network and comprising a user interface configured to allow a user to enter one or more urine parameters of the user;
a processing unit configured to operate on the user device, wherein the processing unit is configured to receive the one or more urine parameters of the user when provided by the user, and to transmit the urine parameters to the server;
a care provider device configured to communicate with the server via the data network and configured to receive the one or more urine parameters from the server; and
a prescription unit configured to generate and transmit, to the user device, a prescription comprising a recommendation that relates to a hydration fluid intake by the user, wherein the prescription is generated by the prescription unit based on the one or more urine parameters transmitted from the user device to the server.

2. The system of claim 1, wherein the one or more urine parameters are selected from the group consisting of; urine volume, urine pH, urinary specific gravity, urine color, a urine biomarker of kidney injury, a urine biomarker of infection, a urine biomarker of stress, a urine biomarker of inflammation, and any combination thereof.

3. The system of claim 1, wherein the one or more urine parameters comprises a volume of urine of less than 2.5 L in 24 h, and wherein the prescription comprises a recommendation of an increase of water intake.

4. The system of claim 1, wherein the one or more urine parameters comprises a volume of urine of greater than 2.5L in 24 h, and wherein the prescription comprises a recommendation of no change in water intake.

5. The system of claim 1, wherein the one or more user's urine parameters comprises a urine color of a score greater than 3 in an Armstrong scale, and wherein the prescription comprises a recommendation of an increase of water intake.

6. The system of claim 1, wherein the one or more urine parameters comprises a urine color of a score less than 4 in an Armstrong scale, and wherein the prescription comprises a recommendation of no change in water intake.

7. The system of claim 1, further comprising an alarm unit configured to provide an alert when one or more of the following urine parameter conditions are met:
a daily urine volume is less than 3.0 L/24 h, less than 2.8 L/24 h, less than 2.6 L/24 h, less than 2.5 L/24 h, less than 2.4 L/24 h, less than 2.2 L/24 h, or less than 2.0 L/24 h;
a urinary specific gravity is greater than 1.010, higher greater than 1.020, or greater than 1.030;
a urine pH is outside the range from 5.0 to 7.0, or outside the range from 5.5 to 6.5;
a urine color of a score greater than 3 in an Armstrong scale.

8. The system of claim 1, wherein the prescription generated by the prescription unit further comprises a recommendation that relates to an intake of a therapeutic substance by the user.

9. The system of claim 1, wherein the prescription generated by the prescription unit is communicated by the care provider device to the user device via the data network.

10. The system of claim 1, further comprising a hydration fluid delivery article configured to communicate with the user device.

11. The system of claim 1, wherein the processing unit is further configured to establish a hydration fluid consumption plan that comprises a plurality of hydration fluid drinking events scheduled at predetermined intervals, wherein each hydration fluid drinking event is associated with a specific hydration fluid intake target, and wherein the processing unit is further configured to compare the hydration fluid intake with the specific hydration fluid intake target for each hydration fluid consumption event and to configure the prescription unit to generate a further recommendation for the user to consume more hydration fluid, as part of the prescription, when the hydration fluid intake during the hydration fluid consumption event is less than the specific hydration fluid intake target associated with the hydration fluid consumption event, and wherein the prescription is provided before a next hydration fluid consumption event.

12. The system of claim 1, wherein the prescription unit is further configured to generate a revised prescription based on an adherence of the user to a hydration fluid consumption plan.

13. The system of claim 1, comprising a non-transitory computer-readable storage medium having stored thereon instructions which, when executed by one or more processing units of the system, cause the one or more processing units to operate, wherein the instructions comprise:
a learning module,
an education module,
a self-motivation module,
a reward module,
a quality-of-life assessment module,
a diet recommendation module, or
any combination thereof.

14. The system of claim 1, further comprising an additional connected device configured to monitor blood pressure, glycemia, body composition, or any combination thereof of the user.

15. The system of claim 14, wherein the additional connected device is in communication with the server.

16. The system of claim 14, wherein the care provider device is configured to receive data provided by the additional connected device.

17. The system of claim 14, wherein the prescription is based on data monitored and transmitted by the additional connected device.

18. The system of claim 1, wherein the prescription unit is configured as a microcomputer as at least part of the care provider device.

19. A method for remotely monitoring a user's hydration state, the method comprising:
providing the system of claim 1;
prompting the user, from the user interface of the user device, to enter the one or more urine parameters;
receiving the one or more urine parameters; and
generating, by the prescription unit, the prescription;
optionally wherein the user has a mineral metabolism disorder, kidney stones, nephrolithiasis, heart disease, chronic kidney disease (CKD), advanced stage kidney failure, advanced stage kidney failure post-extra-renal epuration or hemodialysis or peritoneal dialysis or transplantation, pregnancy or breastfeeding, or is a geriatric or pediatric patient.

20. (canceled)

21. A memory or data storage unit having stored thereon software instructions which, when executed by a processing unit, configure a computer to generate a prescription that comprises a recommendation that relates to a hydration fluid intake suitable for a user, wherein the prescription is based on one or more urine parameters about the user, optionally wherein the prescription further comprises a further recommendation that relates to intake of a therapeutic substance by the user.

22. (canceled)
